(19) United States
(12) Patent Application Publication
(10) Pub. No.: US 2008/0263900 A1
(43) Pub. Date: Oct. 30, 2008

(54) Therapeutic Shoe

(75) Inventors: Patrice Determe, Toulouse (FR); Olivier Laffenetre, Bordeaux (FR); Christophe Cermolacce, Marseille (FR); Jean-Yves Coillard, Saint Cyr au Mont d'Or (FR)

Correspondence Address: YOUNG & THOMPSON, 209 Madison Street, Suite 500, ALEXANDRIA, VA, 22314, US

(73) Assignee: DJO France, Mouguerre (FR)

(21) Appl. No.: 12/097,049

(22) PCT Filed: Dec. 13, 2006
(86) PCT No.: PCT/FR2006/002721

§ 371 Date: Jun. 12, 2008

(30) Foreign Application Priority Data
Dec. 14, 2005 (FR) 0512671

(51) Int. Cl. A43B 23/00; A61F 5/01; A43C 15/00; A61F 5/00

(52) U.S. Cl. 36/107; 36/110; 36/59 C; 602/23

(57) ABSTRACT

The invention relates to a therapeutic shoe which comprises a sole assembly having a tread surface (6) having a curved profile in a sagittal plane of the shoe and an upper surface (17) which comprises a location for the toes and a location for the heel. The sole assembly of the therapeutic shoe according to the invention is longitudinally flexible in the sagittal plane and is shaped so that the curved profile of the tread surface (6) extends opposite the location for the heel and the location for the toes. Furthermore, the therapeutic shoe according to the invention comprises at least one removable rigid reinforcement component (15) which acts counter to the longitudinal flexion of the sole assembly in a sagittal plane.

The invention relates to a therapeutic shoe comprising a sole assembly which has a tread surface which has a curved profile in a sagittal plane of the shoe and an upper surface which comprises a location for the toes and a location for the heel.

The invention relates in particular to post-operative and post-trauma shoes.

There are already known therapeutic shoes which are provided with a rigid sole, one portion of which has a curved profile in a sagittal plane of the shoe which is intended to allow a gradual tilting of the heel towards the front of the foot of the wearer when walking. Compared with a planar rigid sole, a sole of this type facilitates walking and allows a more gradual transfer of the weight of the body from the heel towards the front of the foot when striding. These shoes are suitable for preventing pressure from being applied to the toes of the wearer at the end of the stride.

These shoes are used in particular to prevent any flexion of the front of the foot which may impede recovery following surgery. These shoes in particular allow movement of the phalanxes relative to the metatarsals to be prevented following an operation for correction of a HALLUX VALGUS. These shoes allow any flexion of the toes relative to the metatarsals to be prevented.

There is a requirement to increase the level of comfort for the patient by allowing measured flexion of the foot during walking when it is provided with the therapeutic shoe, in particular in the region of the joints of the metatarsals and the phalanxes.

Furthermore, the rigid immobilisation of the foot is most often followed by a period of re-education during which the patient is encouraged to gradually regain normal use of his foot, or at least regain some of the possibilities for movement of the foot in terms of flexion. The therapeutic shoe is then removed in order to allow, with the assistance of a physiotherapist, flexion movements of the foot to be carried out which enable this re-education.

EP 1 488 715 proposes a post-operative shoe which can be modified and which can be adapted for the different phases following the operation until the patient is able to wear a conventional shoe again. This post-operative shoe comprises a base body with a semi-rigid sole and a set of removable and interchangeable accessories below the sole, in particular a metal strengthening plate and a rounded pad. During a first phase of re-education immediately following the surgical operation on the foot, the post-operative shoe is strengthened using the metal plate which is applied and fixed against the lower face of the sole of the base body. During this phase, the pad is fixed below the metal plate. During a second phase of re-education, the metal plate and the pad in particular are disassembled in order to restore the original flexibility to the sole of the base body. This shoe cannot be readily adapted by the patient himself in accordance with the various phases of re-education. Furthermore, the inventors have established that it does not adequately comply with the actual requirements for immobilisation and mobility encountered during each of the phases.

In this regard, the inventors have established that there is a requirement to allow patients to carry out this re-education themselves, in particular whilst performing common activities carried out by a patient. To this end, it is advantageous to provide therapeutic shoes which promote, to a reduced degree, the carrying out of the flexions of the foot that occur naturally during walking. In particular, it must be possible to cause the patient to carry out, naturally when striding, a movement of the phalanxes relative to the metatarsals with a reduced degree of movement of these bones compared with a movement which occurs during striding when feet are wearing shoes having a flat base.

Furthermore, the inventors have established that it is advantageous to allow the patient to select the time at which to carry out this re-education whilst allowing him at any time to permit or prevent the corresponding flexions of the foot himself. In particular, the intention is for the patient to be allowed to immobilise his foot in response to a feeling of discomfort, pain or fatigue, et cetera.

The object of the present invention is to provide a therapeutic shoe which meets the requirements described above.

More specifically, the object of the invention is to provide at a reduced cost price such a therapeutic shoe which allows such a re-education operation to be carried out independently.

Furthermore, the object of the invention is to provide such a therapeutic shoe whose use is not likely to contradict medical advice in the context of scarring of the foot following surgery or treatment of the majority of injuries and conditions of the foot for which a re-education as described above is recommended.

The invention is further intended to provide a therapeutic shoe of this type which is simple to use, in particular with respect to the operations which have to be carried out in order to allow, prevent or adjust the possibilities for flexion of the foot.

To this end, the invention relates to a therapeutic shoe comprising a sole assembly which has:
a tread surface having a curved profile in a sagittal plane of the shoe,
an upper surface which comprises a location for the toes and a location for the heel,
   characterised in that:
the sole assembly is shaped so that the curved profile of the tread surface extends opposite the location for the heel and the location for the toes,
the sole assembly is longitudinally flexible in the sagittal plane,
the shoe comprises at least one removable rigid reinforcement component which acts counter to the longitudinal flexion of the sole assembly in a sagittal plane.

The therapeutic shoe according to the invention used without any rigid component has the advantage, compared with conventional therapeutic shoes with semi-rigid sole assemblies and planar tread surfaces conventionally used during the re-education phase, of allowing the user to perform a natural striding action with reduced flexion of the foot, which is therefore less painful and/or uncomfortable, so that the re-education of the foot can be carried out more gradually and more naturally for the patient.

Since the movement of the re-education corresponds to a natural movement for the patient, the patient is capable of being able to carry it out in an independent manner.

Still with the therapeutic shoe being used without any rigid component, the therapeutic shoe according to the invention allows weight to be transferred to the front of the foot during a final phase of the movement of the foot during a striding action, producing a progressive flexion of the foot in the region of the joints of the metatarsals and the phalanxes of the foot, to the desired extent.

Furthermore, when the shoe is used in a state provided with the rigid component(s), the therapeutic shoe allows a progressive movement of the foot from the heel to the toes, thus facilitating the striding action.

Advantageously and according to the invention, the tread surface is exclusively constituted by a wear surface of an outer sole which is formed in one piece and which extends opposite a rear end of the location for the heel, as far as a location opposite a front end of the location for the toes.

Advantageously and according to the invention, the curved profile of the tread surface extends as far as a location opposite a front end of the location for the toes.

Advantageously and according to the invention, at least one rigid component is shaped so as to extend opposite the location for the heel and the location for the toes.

Preferably, at least one rigid component is shaped so as to extend as far as a location opposite a front end of the location for the toes. In this manner, the therapeutic shoe is capable of acting counter to a flexion of the foot in the region of the joints of the phalanxes and the metatarsals.

Advantageously and according to the invention, the sole assembly is fixed in a non-removable manner to an upper of the therapeutic shoe.

Advantageously and according to the invention, the tread surface is formed by an outer sole of the sole assembly which forms a lower outer coating of the sole assembly.

Advantageously and according to the invention, at least one rigid reinforcement component is accommodated in a removable manner in a housing of the sole assembly having a shape which corresponds to that of this rigid reinforcement component. Advantageously and according to the invention, this housing is formed in an outer sole of the sole assembly and/or in a removable inner sole of the sole assembly which rests at least partially on an upper inner face of the outer sole, which is opposite the tread surface. Advantageously and according to the invention, the housing is formed by a recess of the inner face of the outer sole. Advantageously and in accordance with the invention, the housing is formed in an insole of the shoe (in the whole of the text, the term “insole” is intended to refer to an upper sole of the shoe which is intended to receive the foot of the user and which therefore has the upper surface of the sole assembly which forms the location for the toes and the location for the heel).

Advantageously and according to the invention, the sole assembly comprises means for removably fixing the inner sole relative to the inner face of the outer sole, which means are capable of acting counter to any relative longitudinal sliding action of the inner sole relative to the inner face of the outer sole.

Advantageously and according to the invention, the outer sole has at least one stud which extends upwards, each stud being capable of extending into a cavity which is provided in the insole, the stud(s) and the cavity or cavities having corresponding shapes in order to form removable fixing means.

Advantageously and according to the invention, each rigid reinforcement component is formed by a resin which is charged with glass fibre.

In a preferred embodiment, advantageously and according to the invention, the shoe comprises a single rigid removable reinforcement component in the form of a plate which extends longitudinally and horizontally opposite the main portion of the tread surface. Advantageously and according to the invention, this plate also extends opposite the main portion of the upper surface of the sole assembly. Advantageously and according to the invention, this rigid reinforcement plate extends longitudinally from the location for the heel to the location for the toes, in particular as far as the front end of the location for the toes. In a variant, there may be provided a rigid reinforcement plate which extends longitudinally below the location for the toes but without reaching the front end of this location for the toes. The same shoe according to the invention may further be provided with two rigid reinforcement plates of different lengths and/or shapes which may be interchanged, the patient selecting the appropriate reinforcement plate depending on the state of his re-education. Preferably, the plate is curved in accordance with an arced profile with a curvature in the same direction as the curved profile of the tread surface. Preferably and according to the invention, the curvature of the plate at least substantially corresponds to that of the curved profile of the tread surface, that is to say, has a similar curved shape.

The invention also relates to a therapeutic shoe which is characterised in combination by all or some of the features mentioned above or below.

Other features, objectives and advantages of the invention will be appreciated from a reading of the following description, given with reference to the appended Figures, in which:

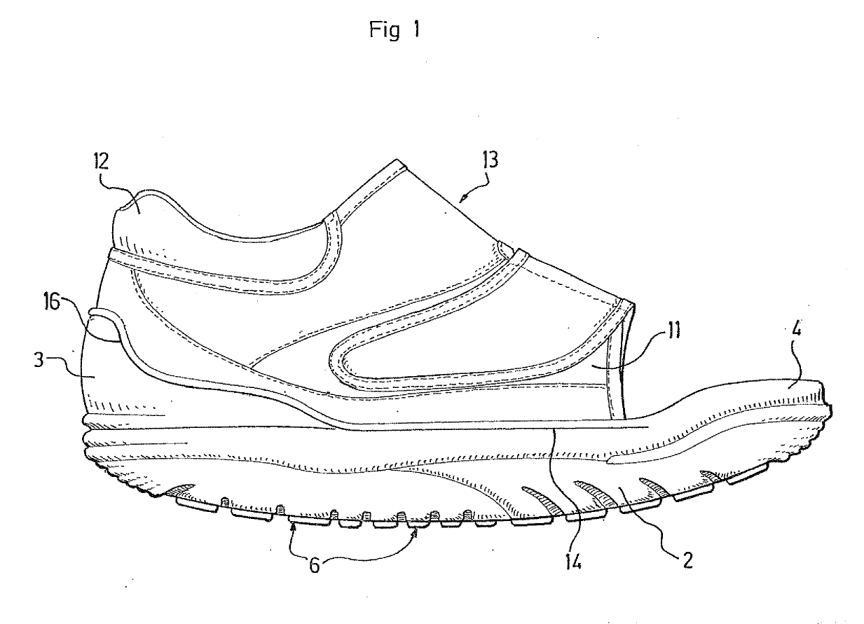

FIG. 1 is a schematic side view of a shoe according to a first embodiment of the invention,

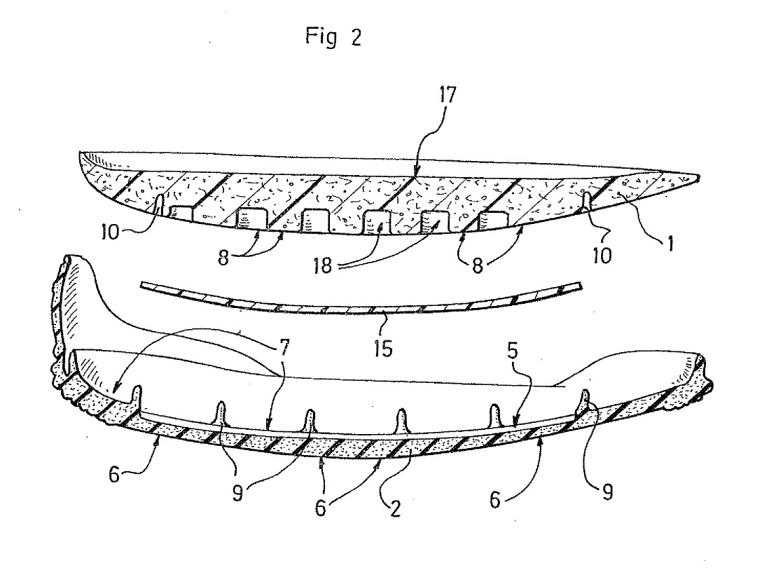

FIG. 2 is an exploded schematic section of a sole assembly of the shoe of FIG. 1, taken along a sagittal plane of the shoe,

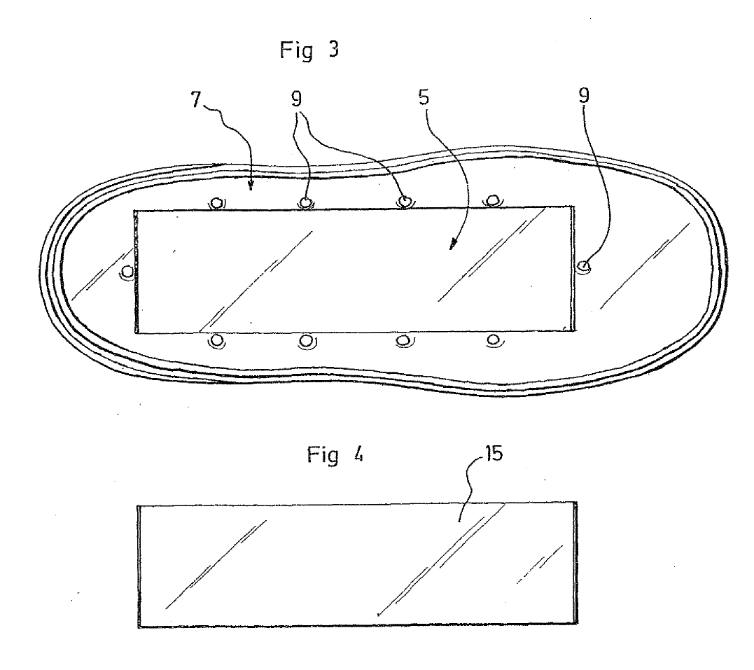

FIG. 3 is a schematic plan view of an outer sole of the sole assembly of FIG. 2,

FIG. 4 is a schematic plan view of a removable rigid reinforcement component of the sole assembly of FIG. 2,

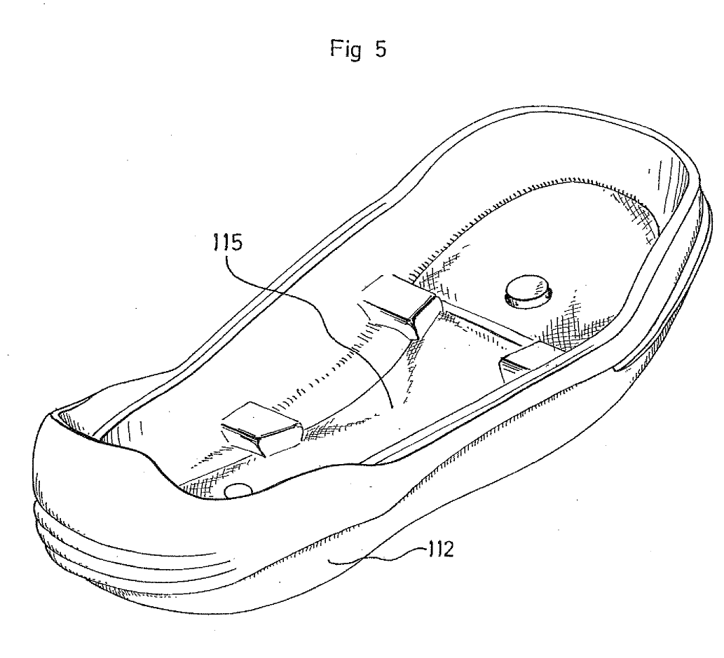

FIG. 5 is a schematic perspective view of an outer sole used to produce a therapeutic shoe according to a second embodiment of the invention.

Hereinafter, the directions and the dimensions must be interpreted, unless indicated otherwise, in accordance with an illustration of the shoe viewed from the front, with the shoe being worn and resting on horizontal ground, with a tread surface of the therapeutic shoe in contact with the ground.

In the preferred embodiment illustrated in FIGS. 1 to 4, the therapeutic shoe according to the invention comprises an upper and a sole assembly which is assembled so as to be fixedly joined to the upper.

The upper is formed by an assembly of textile components which form in particular two regions which are suitable for covering, when the shoe is worn, the instep and the heel of the wearer.

The regions of the upper are capable of moving apart from each other in order to allow the foot of the wearer to be inserted vertically into the shoe and removed vertically from the shoe. The regions extend from the sole assembly so as to meet above the instep of the wearer when the shoe is closed. Each region 11 comprises a flange 13 which extends the region 11 from the junction of the two regions 11 above the instep of the wearer in order to fold down, when the shoe is closed, over the other region 11. The shoe also comprises means for fitting by means of hooks and loops which are capable of allowing the shoe to be closed by one face of the flange 13 of each region 11 being applied to an outer face of the other region 11. One of the flanges extends an upper portion of the region 11 which corresponds to the region of the instep, whilst the other flange 13 extends a lower portion of the region 11 which corresponds to the region of the instep.

Preferably and as illustrated, the regions of the upper extend below the malleolus of the wearer when the shoe is worn. An edge of the upper in the region of the malleolus has a padding 12 in order to provide comfort. In the same manner, the inner side of the upper may be covered with a padding to provide comfort.

In the first embodiment illustrated, the upper of the shoe does not comprise a front overshoe (vamp) so that the shoe has an opening which is arranged at the front of the shoe. An opening of this type, when the shoe is worn, allows access to the toes of the wearer without having to remove the shoe.

In practice, the textile components which form the regions 11 of the upper may be formed by any textile which is capable of retaining the foot in position relative to the sole assembly when walking. Materials other than textiles may obviously be used, for example, leather, or any other appropriate material, in particular a light material in order to provide a satisfactory level of comfort for walking.

The upper as described allows the ankle and the whole of the rear of the foot to be stabilised.

Preferably and as illustrated, the sole assembly comprises an outer sole 2 and an insole 1 which is intended to receive the foot of the patient.

The insole 1 comprises a lower face 8 having a curved profile in a sagittal plane of the sole assembly.

Furthermore, the insole 1 comprises an upper face 17 on which the foot of the wearer rests when the shoe is worn. This upper face 17 may be specifically shaped in accordance with the treatment to be carried out and the ergonomic constraints linked to this treatment. By way of example, as illustrated in the Figures, the upper face 17 of the insole 1 may be generally planar.

Furthermore, the lower face 8 of the insole 1 comprises recesses 18 in order to reduce the weight of the insole 1.

The insole 1 is formed, for example, from expanded polyvinyl chloride. In practice, the insole 1 may be cut from a block of expanded polyvinyl chloride. The insole 1 may be made from any other semi-rigid material which is suitable for medical use, in particular a light material in order to provide a satisfactory level of comfort for walking.

It should be noted that the expression “semi-rigid” is intended to refer to the quality of a material in terms of having mechanical properties under stresses (rigidity, resilience in particular) such that the material is capable of allowing, under the stresses transmitted by the foot during the striding action under normal walking conditions, a flexion of the sole assembly which can be seen in a flexion of the foot which can be sensed by the wearer.

The insole 1 may be made from any light material which is suitable for medical use and which has semi-rigid characteristics in terms of flexion which are suitable for being able to form an insole 1 which allows flexions of reduced extent for the foot during walking when the shoe is used without a removable rigid reinforcement component 15.

Furthermore, the insole 1 may be produced using any other method of shaping which allows the desired shape to be conferred thereon. In particular, the insole may be moulded or extruded, et cetera.

In the first embodiment, the outer sole 2 forms a lower coating of the entire lower face 8 of the insole 1, corresponding to this lower face 8. In this manner, the tread surface 6 of the outer sole 2 and the lower face 8 have similar profiles.

The outer sole 2 has a tread surface 6 having a profile which is curved in a convex manner in the sagittal plane of the sole assembly.

Preferably, the tread surface 6 has a regular profile which is convex and symmetrical at one side and the other of the sagittal plane along planes of section which are parallel with the sagittal plane in order to provide lateral stability for the foot of the patient and allow use for both the left foot and the right foot.

In the first preferred embodiment, the outer sole 2 comprises a concave upper inner face 7 against which the convex lower face 8 of the insole 1 comes into contact. This inner face 7 comprises a recess 5 which is shaped in order to accommodate a rigid bar 15 which has, in a sagittal cross-section of the shoe, an arced profile whose curvature corresponds to the curved profile of the lower face 8 of the insole 1. Preferably, the recess 5 and the rigid bar 15 have corresponding shapes.

The bar 15 may be formed from any rigid material, such as a metal, a polymer or a composite material. By way of example, this material may be polyester charged with glass fibre. The rigid bar 15 may be made from any other rigid material which is suitable for medical use, in particular a light material in order to provide a satisfactory level of comfort for walking.

Furthermore, the inner face 7 of the outer sole 2 advantageously has studs (not illustrated) which are arranged at the edge of the recess 5 extending so as to project horizontally towards the inner side of this edge and allowing the rigid bar 15 to be retained in the recess 5 following its insertion in this recess 5. As an alternative, the studs can be replaced by the edge of the recess 5 being overlapped.

In the first preferred embodiment, the recess 5 is arranged on the inner face 7 of the outer sole 2 so as to extend longitudinally along a sagittal plane of the shoe. The recess 5 and the rigid bar 15 are preferably capable of extending longitudinally when the shoe is worn with the rigid component 15 arranged in the sole assembly, under the foot of the wearer, this rigid component 15 extending opposite the heel and extending towards the front of the foot as far as a location opposite the toes of the wearer and preferably as far as a location opposite the front end of the toes. Consequently, the therapeutic shoe worn in this manner prevents untimely flexion of the foot in the region of the joints of the phalanxes and the metatarsals.

In the first preferred embodiment of the invention, the outer sole 2 has studs 9 which extend upwards from the inner face 7 of the outer sole 2 so as to extend through corresponding cavities 10 which are provided in the lower face 8 of the insole 1. The studs 9 are arranged around the recess 5. These studs 9 facilitate assembly and allow any longitudinal sliding of the insole 1 to be prevented on the inner face 7 of the outer sole 2. The studs 9 also form means for removably fixing the inner sole 1 to the inner face 7 of the outer sole 2. It should be noted that these removable fixing means can be produced using any other appropriate means for removable fixing such as: other means for fixing by means of mutual nesting engagement, fixing means with hooks and loops, straps for fastening the insole to the outer sole, et cetera.

Preferably, the removable fixing means are structurally adapted so as not to significantly influence the flexibility of the sole assembly, this resulting substantially from the shape and the selection of the materials of the outer sole and the insole.

In the first preferred embodiment, the sole assembly is fitted to the upper by means of stitching 14 which extends along a lower edge (not illustrated) of the two regions 11 of the upper, extending over this edge and the thickness of the outer sole 2.

Furthermore, stitching 16 preferably extends along an edge of a welting of the shoe which is formed by an extension of the outer sole 2 which covers a portion of the upper in the region of the heel of the wearer. The outer sole 2 also has an extension which extends opposite the toes of the wearer. This extension forms an impact absorber 4.

In the embodiment of the invention illustrated, it is possible to access the arced rigid bar 15 of the shoe after moving apart the regions 11 of the upper and after having removed the insole 1. Pressure applied to the tread surface 6 of the outer sole 2 allows the arced bar 15 to be removed from the recess 5.

When the shoe is worn without the rigid bar 15 and after the insole 1 has been replaced on the inner face 7 of the outer sole 2, the therapeutic shoe allows a flexion of the foot whilst facilitating a movement of the foot. The extent of flexion provided by the shoe during the striding action depends on the overall structural rigidity of the shoe without the reinforcement component 15. It depends quite particularly on the flexibility of the insole 1 and the outer sole 2.

The therapeutic shoe according to the preferred embodiment of the invention allows rigid support in terms of flexion to be provided for the foot of the wearer when the arced rigid bar 15 is arranged in the recess 5, or more flexible support in terms of flexion for the foot of the wearer when the bar is removed from the sole assembly.

In a variant which is not illustrated, the sole assembly of the therapeutic shoe may be formed by a single sole which forms an outer sole on which the foot of the wearer rests when the shoe is worn.

Furthermore, in a variant which is not illustrated, the therapeutic shoe according to the invention may comprise a plurality of removable rigid reinforcement components, each of these components being accommodated in a removable manner in the sole assembly of the therapeutic shoe in accordance with an arrangement allowing the component to act counter to the flexion of the sole assembly in at least one direction.

Each of the rigid removable reinforcement components may be formed by a rigid bar which may or may not be arced, by a rigid shaft or may comprise any other structure which is capable of acting counter to the flexion of the sole assembly of the shoe in at least one direction.

In a variant which is not illustrated or in combination, there is nothing to prevent a removable rigid reinforcement component from being accommodated in an inner sole rather than in the outer sole. In particular, a removable rigid reinforcement component may be accommodated in an insole 1 of the sole assembly, or in an intermediate sole which is arranged between the insole and the outer sole of the sole assembly, or in any other inner sole of the sole assembly.

In a variant which is not illustrated or in combination, there is nothing to prevent a removable rigid reinforcement component 15 from being accommodated in a groove which is provided in the tread surface of the sole assembly.

In a variant which is not illustrated or in combination, there is nothing to prevent a removable reinforcement component 15 from being formed by a rigid member which is fixed laterally on a sole assembly or a rigid structure which may form a surround for the sole assembly.

FIG. 5 illustrates an outer sole of a therapeutic shoe according to a second embodiment of the invention, similar to that of the first embodiment of the invention except that the removable rigid reinforcement component 115 is accommodated between an inner face of the outer sole 112 and an insole (not illustrated) rather than in a recess of the outer sole. The rigid reinforcement component 115 has a shape which corresponds to that of the inner face so as to extend further in the width dimension of the shoe in the region of a front portion of the shoe. Furthermore, the rigid component extends further in the length dimension of the shoe, that is to say, opposite a heel location of an upper face of the sole assembly and as far as a location opposite a front end of a toe location of the upper face of the sole assembly. The therapeutic shoe according to the improved embodiment allows even more effective immobilisation of the foot to be brought about against flexions of the front of the foot in the longitudinal direction and in terms of lateral twisting.

The invention may involve other construction variants compared with the embodiments illustrated in the Figures and described above by way of non-limiting example.

1-12. (canceled)

13. A therapeutic shoe comprising a sole assembly which has:
a tread surface having a curved profile in a sagittal plane of the shoe,
an upper surface which comprises a location for the toes and a location for the heel,
wherein:
the sole assembly is shaped so that the curved profile of the tread surface extends opposite the location for the heel and the location for the toes,
the sole assembly is longitudinally flexible in the sagittal plane,
the shoe comprises at least one removable rigid reinforcement component which acts counter to the longitudinal flexion of the sole assembly in a sagittal plane.

14. A therapeutic shoe as claimed in claim 13, wherein the curved profile of the tread surface extends as far as a location opposite a front end of the location for the toes.

15. A therapeutic shoe as claimed in claim 13, wherein at least one rigid component is shaped so as to extend opposite the location for the heel and the location for the toes.

16. A therapeutic shoe as claimed in claim 14, wherein at least one rigid component is shaped so as to extend opposite the location for the heel and the location for the toes.

17. A shoe as claimed in claim 13, wherein at least one rigid reinforcement component is accommodated in a removable manner in a housing of the sole assembly having a shape which corresponds to that of the rigid reinforcement component.

18. A shoe as claimed in claim 14, wherein at least one rigid reinforcement component is accommodated in a removable manner in a housing of the sole assembly having a shape which corresponds to that of the rigid reinforcement component.

19. A shoe as claimed in claim 15, wherein at least one rigid reinforcement component is accommodated in a removable manner in a housing of the sole assembly having a shape which corresponds to that of the rigid reinforcement component.

20. A shoe as claimed in claim 17, wherein the housing is formed in an outer sole of the sole assembly and/or in a removable inner sole of the sole assembly which rests at least partially on an upper inner face of the outer sole which is opposite the tread surface.

21. A shoe as claimed in claim 20, wherein the housing is formed by a recess of the inner face.

22. A shoe as claimed in either claim 20, wherein the housing is formed in an insole of the shoe.

23. A shoe as claimed in either claim 21, wherein the housing is formed in an insole of the shoe.

24. A shoe as claimed in claim 20, wherein the sole assembly comprises means for removably fixing the inner sole relative to the inner face of the outer sole, which means are capable of acting counter to any relative longitudinal sliding action of the inner sole relative to the inner face of the outer sole.

25. A shoe as claimed in claim 21, wherein the sole assembly comprises means for removably fixing the inner sole relative to the inner face of the outer sole, which means are capable of acting counter to any relative longitudinal sliding action of the inner sole relative to the inner face of the outer sole.

26. A shoe as claimed in claim 22, wherein the sole assembly comprises means for removably fixing the inner sole relative to the inner face of the outer sole, which means are capable of acting counter to any relative longitudinal sliding action of the inner sole relative to the inner face of the outer sole.

27. A shoe as claimed in claim 24, wherein the outer sole has at least one stud which extends upwards, each stud being capable of extending into a cavity which is provided in the insole, the stud(s) and the cavity or cavities having corresponding shapes in order to form removable fixing means.

28. A shoe as claimed in claim 13, wherein it comprises a single rigid removable reinforcement component in the form of a plate which extends longitudinally and horizontally opposite the main portion of the tread surface.

29. A shoe as claimed in claim 28, wherein the plate is curved in accordance with an arced profile with a curvature in the same direction as the curved profile of the tread surface.

30. A therapeutic shoe as claimed in claim 13, wherein the tread surface is exclusively constituted by a tread surface of an outer sole which is formed in one piece and which extends opposite a rear end of the location for the heel, as far as a location opposite a front end of the location for the toes.
